(19) United States
(12) Patent Application Publication
(10) Pub. No.: US 2011/0318293 A1
(43) Pub. Date: Dec. 29, 2011

(54) Hair Preparation Comprising Royal Jelly

(76) Inventors: Astrid Kleen, Hamburg (DE); Anja Reichert, Duesseldorf (DE); Christa Hartwich, Elmshorn (DE); Stephan Schwartz, Wedel (DE)

(21) Appl. No.: 13/228,538

(22) Filed: Sep. 9, 2011

Related U.S. Application Data
(63) Continuation of application No. PCT/EP10/52663, filed on Mar. 3, 2010.

(30) Foreign Application Priority Data
Mar. 11, 2009 (DE) 10 2009 001 484.5

(51) Int. Cl. A61K 8/98; A61Q 5/10; A61Q 5/04

(52) U.S. Cl. 424/70.2; 132/212; 8/406

(57) ABSTRACT

Agent for changing the color and/or permanently changing the shape of keratinic fibers, wherein the agent contains in a cosmetic carrier at least one color- and/or shape-changing component as well as an animal-based care agent, in particular royal jelly, in combination with a polyalkoxylated fatty substance. The agents are suitable for reducing hair damage which occurs in connection with oxidative hair treatment such as oxidative hair coloring, hair lightening and perming.

### CROSS REFERENCE TO RELATED APPLICATIONS

This application is a continuation of International Patent Application No. PCT/EP2010/052663 filed 3 Mar. 2010, which claims priority to German Patent Application No. 10 2009 001 484.5 filed 11 Mar. 2009, both of which are incorporated herein by reference.

The invention relates to an agent for changing the color and/or permanently changing the shape of keratinic fibers, wherein the agent contains in a cosmetic carrier at least one color- and/or shape-changing component as well as an animal-based care agent, in particular royal jelly, in combination with a polyalkoxylated fatty substance. Agents according to the invention are suitable for reducing hair damage which occurs in connection with oxidative hair treatment such as oxidative hair coloring, hair lightening and penning. The invention therefore relates to a method for applying such agents to keratin-containing fibers. The invention furthermore relates to use of the agent for coloring and/or for permanent shape changing to reduce hair damage.

Changing the shape and color of hair is an important area of modern cosmetics. It allows the appearance of the hair to be adapted both to the latest fashion trends and to the personal preferences of the individual. Not only should the agents have the desired coloring and shaping capacity, they should minimize possible damage to the hair and preferably also have additional care properties.

One skilled in the art is familiar with various coloring systems for providing color-changing cosmetic agents, particularly for skin or keratin-containing fibers such as human hair, depending on the requirements of the coloring process.

For permanent, intense colors with corresponding fastness properties, oxidation coloring agents are used. Such coloring agents typically contain oxidation dye precursors known as developer components and coupler components, which in the presence of oxidizing agents or atmospheric oxygen and one another form the actual dyes. Oxidation coloring agents produce outstanding, long-lasting coloring results. For temporary colors, coloring or tinting agents containing substantive dyes as the coloring component are typically used. These are dye molecules which attach directly to the substrate and require no oxidative process to develop the color. Finally, another coloring method which has attracted great attention is where precursors of the natural hair dye melanin are applied to the substrate (e.g., hair), whereupon through oxidative processes they develop nature-analogous dyes in the hair.

If substrates are to be lightened or bleached, the dyes coloring the substrate can be mostly decolorized by oxidation using corresponding oxidizing agents, such as hydrogen peroxide.

The lasting shaping of keratin fibers is conventionally brought about by mechanically shaping the fibers and fixing the shape by means of suitable auxiliary agents. Before and/or after this shaping, the fibers are treated with an aqueous preparation of a keratin-reducing substance that cleaves a part of the disulfide bonds of the keratin to form thiol groups, resulting in a loosening of the peptide crosslinking and a reorientation of the keratin structure as a result of the tightening of the fibers by the mechanical shaping. At the end of a contact period the fibers are rinsed with water or an aqueous solution. In a second step the fibers are then treated with an aqueous preparation of an oxidizing agent under which new disulfide bonds are formed, causing the keratin structure to be fixed in the designated shape. At the end of a contact period this second solution is rinsed out and the fibers are freed from the mechanical shaping aids (rollers, foam curlers). A known method of this type is the permanent waving of human hair. This method can be used both to create curls and waves in smooth hair and to smooth curly hair.

To improve the condition of the fibers, it has long been common practice to subject fibers to a special post-treatment following the color- and/or shape-changing treatment. Here the hair is treated with special active ingredients, for example, quaternary ammonium salts or special polymers, typically in the form of a rinse. Depending on the formulation, combability, hold and fullness of the hair are improved and the number of split ends is reduced by this treatment.

Oxidative hair treatment agents, in spite of their advantageous coloring, lightening and/or shape-changing properties, are associated with disadvantages for the user. Firstly, the use of oxidizing agents can damage the hair structure and surface of the hair. The hair can become brittle, its elasticity decrease and it becomes less easy to comb. This damage increases with the time of application. Secondly, oxidative coloring agents generally require a basic pH range for color generation, in particular from pH 9.0 to pH 10.5. However, the splaying of the external cuticle of the hair associated with the basic pH leads to an unpleasant surface sensitivity of the hair, making it more difficult to comb when wet and dry. For the consumer this results in an increased need to use additional post-treatment agents such as conditioning agents. Further, hair structure is also affected by external environmental influences. These include mechanical and thermal influences, such as combing and blow-drying. Weather influences such as wind, rain and UV radiation in sunlight, and additional external stresses such as chlorinated swimming pool water or perspiration likewise contribute to damage to the hair structure and hair surface.

There is therefore still a need for caring active ingredients for the color- and/or shape-changing treatment of fibers. The present invention therefore provides a color- and/or shape-changing agent by which the aforementioned disadvantages of customary color- and/or shape-changing agents are reduced. Hair that has been affected and that has previously been damaged by external influences should experience as little additional damage as possible through color and/or shape changing by means of color- and/or shape-changing agents. In particular, the color- and/or shape-changing agents should provide protection against oxidative damage to the hair structure and the hair surface. Caring properties of the agents are particularly desirable, such that the user can dispense with the use of additional conditioning and post-treatment agents.

It has now been found that color- and/or shape-changing agents containing, in addition to a color- and/or shape-changing component, at least one polyalkoxylated fatty substance as well as at least one animal-based care component avoid the aforementioned disadvantages. As a result of the protective effect associated with the use of the agent according to the invention, hair damage can be minimized or even a hair care effect achieved.

The invention therefore firstly provides an agent for changing the color and/or shape of keratinic fibers, particularly human hair. The agent contains in a cosmetic carrier at least one color- and/or shape-changing component and additionally contains at least one polyalkoxylated fatty substance and at least one animal-based care component.

“Keratinic fibers” here refers to fur, wool, feathers and human hair. Although agents according to the invention are primarily suitable for dyeing keratin fibers, there is nothing in principle to preclude their use in other fields.

Agents according to the invention contain the active ingredients in a cosmetic carrier. Within the meaning of the invention this cosmetic carrier can be aqueous, alcoholic or aqueous-alcoholic. For the purposes of hair coloring such carriers include creams, emulsions, gels or surfactant-containing foaming solutions, such as shampoos, foam aerosols or other preparations suitable for use on the hair.

Within the meaning of the present invention aqueous-alcoholic carriers refer to water-containing compositions containing 3 to 70 wt. % of a C₁ to C₄ alcohol, relative to the overall weight of the application mixture, such as ethanol or isopropanol. An aqueous carrier contains according to the invention at least 30 wt. %, particularly at least 50 wt. % of water, based on total weight of the application mixture.

Agents according to the invention contain an animal-based care component as a substantial ingredient. This component is preferably constituted by care substances obtained from insect products. Examples of such substances are silk proteins and bee products, such as honey, beeswax and royal jelly.

Preferred agents according to the invention contain royal jelly as the animal-based care component.

Royal jelly is a product derived from honeybees and used by the bees as a special nutrient to raise the queens of the bee population. In addition to water it contains approximately 4 to 5 wt. % lipids, approximately 14-16 wt. % sugars, particularly glucose, fructose and sucrose, approximately 13-14 wt. % of proteins and amino acids, as well as small amounts of vitamins, provitamins and trace elements.

This combination of ingredients allows for the caring effect of royal jelly to occur even when contained in small proportions in the hair treatment agent. Preferred agents therefore contain royal jelly in an amount from 0.00001 to 5%, particularly from 0.0001 to 1%, most particularly preferably from 0.01 to 0.5%, based on total weight of the ready-to-use agent.

As the second substantial ingredient, agents according to the invention contain at least one polyalkoxylated fatty substance. Within the context of the present invention this refers to a fatty substance that has been etherified or esterified with one or more units of ethylene oxide, propylene oxide and/or glycerol via at least one hydroxyl and/or carboxyl group. Examples of such polyalkoxylated fatty substances include—
a) addition products of 4 to 30 mol of ethylene oxide and/or 0 to 5 mol of propylene oxide with linear or branched, saturated or unsaturated fatty alcohols having 8 to 22 C atoms, with linear or branched, saturated or unsaturated fatty acids having 12 to 22 C atoms and with alkyl phenols having 8 to 15 C atoms in the alkyl group;
b) mono- or polyglycerylated linear or branched, saturated or unsaturated fatty alcohols having 8 to 22 C atoms;
c) polyethoxylated fatty acid alkyl esters of the formula RCOO—(C₂H₄O)ₙ—R′, wherein R is a linear or branched, saturated or unsaturated alkyl residue having 7 to 21 C atoms, n is a whole number from 1 to 50, and R′ is an alkyl residue having 1 to 30 C atoms, preferably 1 to 4 C atoms; and
d) addition products of 1 to 100 mol of ethylene oxide with hydroxyl group-containing fatty acid triglycerides, such as castor oil and hydrogenated castor oil.

Preferred saturated and unsaturated fatty alcohols are octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Technical fatty alcohols having 12 to 18 carbon atoms, such as coconut fatty alcohols, palm fatty alcohols, palm kernel fatty alcohols or tallow fatty alcohols, are preferred. Preferred fatty acids are lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, behenic acid and erucic acid and natural and/or technical mixtures thereof.

Preferred agents include those wherein the polyalkoxylated fatty substance is chosen from addition products of polyethylene oxide with fatty alcohols and/or hydroxyl group-containing fatty acid triglycerides and from mono- or polyglycerylated fatty alcohols.

Of these, addition products of polyethylene oxide with fatty alcohols are particularly preferred which have an average degree of ethoxylation of 10 to 45, particularly 12 to 30. The average degree of ethoxylation is understood to be the molar amount of ethylene oxide with which one mole of fatty alcohol was reacted. Examples of such compounds are known under the INCI names Steareth-20, Coceth-15, Oleth-20 or Ceteareth-30.

Examples of mono- or polyglycerylated fatty alcohols are compounds listed under the INCI names Polyglyceryl-4 lauryl ether or Polyglyceryl-2 oleyl ether.

Particularly preferred agents contain as the polyalkoxylated fatty substance a polyalkoxylated, hydroxyl group-containing fatty acid triglyceride, particularly an addition product of polyethylene oxide with hydrogenated and/or unhydrogenated castor oil.

An addition product of polyethylene oxide with hydrogenated castor oil has proved to be most particularly advantageous, wherein the average degree of ethoxylation is from 1 to 100, preferably from 10 to 75, and particularly from 20 to 60.

Preferred examples of such compounds are compounds known under the INCI names PEG-5 hydrogenated castor oil, PEG-7 hydrogenated castor oil, PEG-10 hydrogenated castor oil, PEG-20 hydrogenated castor oil, PEG-25 hydrogenated castor oil, PEG-40 hydrogenated castor oil, PEG-45 hydrogenated castor oil, PEG-54 hydrogenated castor oil and PEG-60 hydrogenated castor oil, PEG-40 hydrogenated castor oil being preferred in particular.

Preferred agents according to the invention contain the polyalkoxylated fatty substance in an amount from 0.001 to 20 wt. %, particularly from 0.01 to 15 wt. %, based on total weight of the ready-to-use agent.

It has emerged that hair damage can be further reduced and care performance improved by addition of a water-soluble organic solvent to the agent according to the invention.

In a preferred embodiment, the agent therefore additionally contains at least one water-soluble, organic solvent. Organic solvents are liquid at room temperature under normal pressure. Preferred organic solvents are C₁-C₆ alcohols optionally bearing further functional groups to improve water solubility, such as 4-methoxybutanol, ethyl diglycol, 1,2-propylene glycol, n-propanol, n-butanol, n-butylene glycol, glycerol, diethylene glycol monoethyl ether, and diethylene glycol mono-n-butyl ether. Preferred water-soluble, organic solvents contain at least two hydroxyl groups. These are preferably chosen from 1,2-ethanediol, 1,2-propanediol (1,2-propylene glycol), 1,3-propanediol, 1,2-butanediol, 1,3-butanediol, 1,4-butanediol, 2,3-butanediol, 1,6-hexanediol, 2-(2-hydroxyethoxy)ethanol and glycerol. 1,2-Propanediol and glycerol are particularly preferred.

Agents according to the invention are used to particular advantage to change the color of keratinic fibers. Preferred agents according to the invention therefore contain as the color-changing component at least one oxidation dye precursor and/or at least one substantive dye and/or at least one precursor of nature-analogous dyes and/or at least one lightening agent.

In a first embodiment the color-changing components are chosen from oxidation dye precursors.

Oxidation dye precursors are preferably used in an amount from 0.005 to 20 wt. %, preferably from 0.05 to 5 wt. % and particularly preferably from 0.1 to 5 wt. %, based on total weight of the ready-to-use oxidation coloring agent.

In a preferred embodiment the agents contain at least one oxidation dye precursor of the developer type and/or coupler type. Coloring agents according to the invention preferably contain at least one oxidation dye precursor of the developer type and at least one oxidation dye precursor of the coupler type.

Developer and coupler components are typically used in free form. For substances containing amino groups, however, it can be preferable to use them in salt form, particularly in the form of hydrochlorides and hydrobromides or sulfates.

Developer components can be chosen from p-phenylenediamine, binuclear developer components, p-aminophenol, o-aminophenol, heterocyclic developer components and/or derivatives of the above classes of substances. The developer components are preferably used in an amount from 0.005 to 20 wt. %, preferably 0.1 to 5 wt. %, based on total weight of the ready-to-use oxidation coloring agent.

Preferred p-phenylenediamines are chosen from one or more compounds of p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(2-hydroxyethyl)-p-phenylenediamine, 4-N,N-bis-(2-hydroxyethyl)amino-2-methylaniline, 4-N,N-bis-(2-hydroxyethyl)amino-2-chloroaniline, 2-(2-hydroxyethyl)-p-phenylenediamine, 2-(1,2-dihydroxyethyl)-p-phenylenediamine, 2-fluoro-p-phenylenediamine, 2-isopropyl-p-phenylenediamine, N-(2-hydroxypropyl)-p-phenylenediamine, 2-hydroxymethyl-p-phenylenediamine, N,N-dimethyl-3-methyl-p-phenylenediamine, N-ethyl-N-2-hydroxyethyl-p-phenylenediamine, N-(2,3-dihydroxypropyl)-p-phenylenediamine, N-(4′-aminophenyl)-p-phenylenediamine, N-phenyl-p-phenylenediamine, 2-(2-hydroxyethyloxy)-p-phenylenediamine, 2-methoxymethyl-p-phenylenediamine, 2-(2-acetylaminoethyloxy)-p-phenylenediamine, N-(2-methoxyethyl)-p-phenylenediamine, N-(4-amino-3-methylphenyl)-N-[3-(1H-imidazol-1-yl)propyl]amine, 5,8-diaminobenzo-1,4-dioxane and the physiologically tolerable salts thereof. Particularly preferred p-phenylenediamine derivatives according to the invention are chosen from at least one compound of p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, 2-methoxymethyl-p-phenylenediamine and the physiologically tolerable salts of these compounds.

It can further be preferred to use as the developer component compounds having at least two aromatic nuclei substituted with amino and/or hydroxyl groups. Preferred binuclear developer components are chosen from at least one of the following compounds: N,N′-bis-(2-hydroxyethyl)-N,N′-bis-(4′-aminophenyl)-1,3-diaminopropan-2-ol, N,N′-bis-(2-hydroxyethyl)-N,N′-bis-(4′-aminophenyl)ethylenediamine, N,N′-bis-(4′-aminophenyl)tetramethylenediamine, N,N′-bis-(2-hydroxyethyl)-N,N′-bis-(4′-aminophenyl)tetramethylenediamine, N,N′-bis-[4-(methylamino)phenyl]tetramethylenediamine, N,N′-diethyl-N,N′-bis-(4′-amino-3′-methylphenyl)ethylenediamine, bis-(2-hydroxy-5-aminophenyl)methane, N,N′-bis-(4′-aminophenyl)-1,4-diazacycloheptane, N,N′-bis-(2-hydroxy-5-aminobenzyl)piperazine, N-(4′-aminophenyl)-p-phenylenediamine and 1,10-bis-(2′,5′-diaminophenyl)-1,4,7,10-tetraoxadecane and the physiologically tolerable salts thereof. Particularly preferred binuclear developer components are selected from N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecane or one of the physiologically tolerable salts of these compounds.

It can also be preferred to use a p-aminophenol derivative or one of its physiologically tolerable salts as the developer component. Preferred p-aminophenols include p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(2-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-[(2-hydroxyethyl)aminomethyl]phenol, 4-amino-2-(1,2-dihydroxyethyl)phenol, 4-amino-2-fluorophenol, 4-amino-2-chlorophenol, 4-amino-2,6-dichlorophenol, 4-amino-2-(diethylaminomethyl)phenol and physiologically tolerable salts thereof. Particularly preferred compounds are p-aminophenol, 4-amino-3-methylphenol, 4-amino-2-aminomethylphenol, 4-amino-2-(1,2-dihydroxyethyl)phenol and 4-amino-2-(diethylaminomethyl)phenol.

The developer component can also be chosen from o-aminophenol and derivatives thereof, such as 2-amino-4-methylphenol, 2-amino-5-methylphenol or 2-amino-4-chlorophenol.

The developer component can furthermore be chosen from heterocyclic developer components, such as pyrimidine derivatives, pyrazole derivatives, pyrazolopyrimidine derivatives and physiologically tolerable salts thereof. Preferred pyrimidine derivatives are the compounds 2,4,5,6-tetraminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyrimidine. Preferred pyrazole derivatives are the compounds selected from 4,5-diamino-1-methylpyrazole, 4,5-diamino-1-(2-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-t-butyl-1-methylpyrazole, 4,5-diamino-1-t-butyl-3-methylpyrazole, 4,5-diamino-1-(2-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxy-methyl-1-isopropylpyrazole, 4,5-diamino-3-methyl-1-isopropylpyrazole, 4-amino-5-(2-aminoethyl)-amino-1,3-dimethylpyrazole, and physiologically tolerable salts thereof.

Preferred pyrazolopyrimidine derivatives include derivatives of pyrazolo[1,5-a]pyrimidine and tautomeric forms thereof, if a tautomeric equilibrium exists. Useful pyrazolo[1,5-a]pyrimidines include pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-aminopyrazolo[1,5-a]pyrimidin-3-yl)-(2-hydroxyethyl)amino]ethanol; 5,6-dimethylpyrazolo[1,5-a]pyrimidin-3,7-diamine, 2,6-dimethylpyrazolo[1,5-a]pyrimidine-3,7-diamine, 3-amino-7-dimethylamino-2,5-dimethylpyrazolo[1,5-a]pyrimidine as well as physiologically tolerable salts thereof and tautomeric forms thereof, if a tautomeric equilibrium exists.

Most particularly preferred developer components are chosen from at least one compound from p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraminopyrimidine, 4-hydroxy-2,5,6-triaminopyrimidine, 2-hydroxy-4,5,6-triaminopyrimidine, and the physiologically tolerable salts of these compounds.

In the context of oxidative dyeing, coupler components develop no significant color on their own but always need the presence of developer components. It is therefore preferable that, with the use of at least one coupler component, at least one developer component is additionally used.

Coupler components according to the invention include m-aminophenol, m-diaminobenzene, o-diaminobenzene, o-aminophenol, naphthalene derivatives having at least one hydroxyl group, di- or trihydroxybenzene, pyridine, pyrimidine, monohydroxy- or monoaminoindole, monohydroxy- or monoaminoindoline, pyrazolone, benzomorpholine, quinoxaline and/or derivatives of the above classes of substances. The coupler components are preferably used in an amount from 0.005 to 20 wt. %, preferably 0.1 to 5 wt. %, based on total weight of the ready-to-use oxidation coloring agent.

Preferred m-aminophenols or derivatives thereof include at least one compound chosen from m-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-diethylaminophenol, N-cyclopentyl-3-aminophenol, 1,3-dihydroxy-5-methylaminobenzene, 3-ethylamino-4-methylphenol, 2,4-dichloro-3-aminophenol and the physiologically tolerable salts of all aforementioned compounds.

Preferred m-diaminobenzenes or derivatives thereof include at least one compound chosen from m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene and the physiologically tolerable salts of the cited compounds.

Preferred o-diaminobenzenes or derivatives thereof include at least one compound chosen formed from 3,4-diaminobenzoic acid and 2,3-diamino-1-methylbenzene and the physiologically tolerable salts of the cited compounds.

Preferred di- or trihydroxybenzenes and derivatives thereof include at least one compound chosen from resorcinol, resorcinol monomethyl ether, 2-methylresorcinol, 5-methylresorcinol, 2,5-dimethylresorcinol, 2-chlororesorcinol, 4-chlororesorcinol, pyrogallol and 1,2,4-trihydroxybenzene.

Preferred pyridine derivatives include at least one compound chosen from 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 3,4-diaminopyridine, 2-(2-methoxyethyl)amino-3-amino-6-methoxypyridine, 2-(4′-methoxyphenyl)amino-3-aminopyridine, and the physiologically tolerable salts of the aforementioned compounds.

Preferred naphthalene derivatives containing at least one hydroxyl group include at least one compound chosen from 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene.

Preferred indole derivatives include at least one compound chosen from 4-hydroxyindole, 6-hydroxyindole and 7-hydroxyindole and the physiologically tolerable salts of the aforementioned compounds.

Preferred indoline derivatives include at least one compound chosen from 4-hydroxyindoline, 6-hydroxyindoline and 7-hydroxyindoline and the physiologically tolerable salts of the aforementioned compounds.

Preferred pyrimidine derivatives include at least one compound chosen from 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine and the physiologically tolerable salts of the aforementioned compounds.

Particularly preferred coupler components according to the invention include 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or the physiologically tolerable salts of the aforementioned compounds.

Developer components and coupler components are generally used in approximately molar amounts to one another. Although the molar use has proved convenient, a certain excess of individual oxidation dye precursors is not disadvantageous, such that developer components and coupler components can be in a molar ratio of 1 to 0.5 to 1 to 3, particularly 1 to 1 to 1 to 2.

Agents according to the invention can also contain at least one substantive dye as the color-changing component. These are dyes which attach directly to the hair and require no oxidative process to develop the color. Substantive dyes are conventionally nitrophenylene diamines, nitroaminophenols, azo dyes, anthraquinones or indophenols. Substantive dyes are preferably used in an amount from 0.001 to 20 wt. %, based on the complete application preparation. The total amount of substantive dyes is preferably at most 20 wt. %. Substantive dyes are known as anionic, cationic and non-ionic substantive dyes.

Preferred anionic substantive dyes include those compounds known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue.

Preferred cationic substantive dyes include cationic triphenylmethane dyes such as Basic Blue 7, Basic Blue 26, Basic Violet 2 and Basic Violet 14, aromatic systems which are substituted with a quaternary nitrogen group, such as Basic Yellow 57, Basic Red 76, Basic Blue 99, Basic Brown 16 and Basic Brown 17, as well as substantive dyes containing a heterocyclic compound having at least one quaternary nitrogen atom, particularly Basic Yellow 87, Basic Orange 31 and Basic Red 51. Cationic substantive dyes sold under the trademark Arianor® are likewise most particularly preferred cationic substantive dyes according to the invention.

Non-ionic nitro and quinone dyes and neutral azo dyes in particular are suitable as non-ionic substantive dyes. Preferred non-ionic substantive dyes include those compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-4-nitrophenol.

In a further embodiment, the color-changing component is chosen from dye precursors of nature-analogous dyes.

Indoles and indolines containing at least two groups chosen from hydroxyl and/or amino groups, preferably as a substituent on the six-membered ring, are preferably used as dye precursors of nature-analogous dyes. These groups can bear further substituents, for example, in the form of an etherification or esterification of the hydroxyl group or an alkylation of the amino group. In a further embodiment the coloring agents contain at least one indole and/or indoline derivative. Compositions according to the invention containing precursors of nature-analogous dyes are preferably used as air-oxidative coloring agents. In this embodiment the compositions are not mixed with an additional oxidizing agent. Dye precursors of nature-analogous dyes are each preferably used in an amount from 0.001 to 5 wt. %, relative to the complete application preparation. The total amount of substantive dyes is preferably at most 3 wt. %.

Preferred indoline derivatives include 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline and 5,6-dihydroxyindoline-2-carboxylic acid, in particular N-methyl-5,6-dihydroxyindoline, N-ethyl-5,6-dihydroxyindoline, N-propyl-5,6-dihydroxyindoline, N-butyl-5,6-dihydroxyindoline and particularly preferably 5,6-dihydroxyindoline.

Preferred indole derivatives include 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, preferably N-methyl-5,6-dihydroxyindole, N-ethyl-5,6-dihydroxyindole, N-propyl-5,6-dihydroxyindole, N-butyl-5,6-dihydroxyindole and in particular 5,6-dihydroxyindole.

It is not necessary for oxidation dye precursors, substantive dyes or nature-analogous dyes to be uniform compounds. Instead, it is possible for the individual dyes to also contain small amounts of further components arising from the manufacturing processes for the individual dyes, provided that they do not adversely influence the dyeing result or need to be excluded for other reasons, for example, toxicological reasons.

In a further embodiment, the color-changing component is chosen from lightening agents. Such lightening agents are typically chemical oxidizing agents, with which natural and artificial dyes in the hair are destroyed, thus bleaching the hair. Persulfates, peroxomonosulfates, chlorites, hypochlorites and particularly hydrogen peroxide or addition products thereof with urea, melamine and sodium borate are therefore suitable as lightening agents.

The lightening effect can also be desired in addition to a coloring. Agents according to the invention can also contain lightening and/or bleaching agents and thus be agents having a simultaneous coloring and lightening action. Such agents are described below as “coloring agents”, as “lightening coloring agents” or as “coloring and lightening agents”.

Hydrogen peroxide is preferably used as the oxidizing agent. The amount of hydrogen peroxide in the ready-to-use agent is preferably 0.5 to 12 wt. %, more preferably 0.8 to 6 wt. %, based on total weight of the ready-to-use agent.

Such oxidizing agent preparations are preferably aqueous, free-flowing oxidizing agent preparations. Preferred preparations are those wherein the free-flowing oxidizing agent preparation—relative to its weight—contains 40 to 90 wt. %, preferably 50 to 85 wt. %, particularly preferably 55 to 80 wt. %, more preferably 60 to 77.5 wt. %, and in particular 65 to 75 wt. % of water.

In the case of oxidative colorings, the color can develop with atmospheric oxygen. A chemical oxidizing agent is preferably used, however, particularly if a lightening effect on human hair is sought in addition to the coloring. According to the invention the color-changing agent as an oxidization coloring agent can also be applied to the hair together with a catalyst, which activates oxidation of the dye precursors, for example, through atmospheric oxygen. Such catalysts include certain enzymes, iodides, quinones or metal ions.

For intense lightening of very dark hair, the use of hydrogen peroxide or its addition products with organic or inorganic compounds alone is often not sufficient. In these cases a combination of hydrogen peroxide and inorganic persulfates is generally used, resulting in an increase in the lightening capacity of the agents. Preferred persulfate salts are ammonium peroxodisulfate, potassium peroxodisulfate and sodium peroxodisulfate.

Peroxodisulfate salts can be included in an amount from 0.1 to 25 wt. %, particularly from 0.5 to 15 wt. %, relative to the overall weight of the ready-to-use agent. Use of persulfate salts or peroxodisulfate salts generally takes place in the form of an optionally dedusted powder or a pressed molding.

If additional oxidizing agents are used, the actual coloring and/or lightening agent is conveniently prepared directly before use by mixing a preparation according to the invention containing in a cosmetic carrier at least one oxidation dye precursor and a preparation containing the additional oxidizing agent, in particular hydrogen peroxide. The agent according to the invention can contain the combination of royal jelly and at least one polyalkoxylated fatty substance in the preparation with oxidation dye precursors and/or in the oxidizing agent preparation. The preparation with oxidation dye precursors preferably contains a combination of royal jelly and at least one polyalkoxylated fatty substance.

If an intense lightening is desired, it is preferable for a lightening preparation containing at least one inorganic persulfate to be additionally incorporated in the oxidizing agent preparation before mixing with the coloring preparation according to the invention.

Agents according to the invention can also be used to change the shape of keratinic fibers. Preferred agents according to the invention therefore contain as the shape-changing component at least one keratin-reducing substance, preferably mercaptans.

Agents for shape-changing typically consist of two or three preparations which are applied to the fibers in succession. The following designations are used below:

“Permanent wave agent” for the aqueous preparation of the keratin-reducing substance,

“Intermediate rinse” for the first rinse, and

“Fixing agent” for the aqueous preparation of the oxidizing agent.

Although the combination of at least one polyalkoxylated fatty substance and at least one animal-based care component can be used in any of the cited preparations, it has proved particularly preferable according to the invention if it is contained in the permanent wave agent.

Permanent wave agents according to the invention obligatorily contain at least one keratin-reducing substance as the shape-changing component, preferably mercaptans. Such compounds include thioglycolic acid, thiolactic acid, thiomalic acid, mercaptoethanesulfonic acid and salts and esters thereof, cysteamine, cysteine, Bunte salts and salts of sulfurous acid. The alkali or ammonium salts of thioglycolic acid and/or thiolactic acid and the free acids are preferably suitable as shape-changing components. These are preferably used in permanent wave agents in concentrations of 0.5 to 1.0 mol/kg with a pH of 5 to 12, particularly 7 to 9.5. To obtain this pH, permanent wave agents according to the invention typically contain alkalizing agents such as ammonia, alkali and ammonium carbonates and hydrogen carbonates or organic amines such as monoethanolamine.

Permanent wave lotions according to the invention can furthermore contain components to strengthen the permanent wave capacity, preferably chosen from heterocyclic compounds such as imidazole, pyrrolidine, piperidine, dioxolane, dioxane, morpholine, piperazine and derivatives of these compounds, in particular 1-methylimidazole, 2-methylimidazole, 4(5)-methylimidazole, 1,2-dimethylimidazole, 2-ethylimidazole, 2-isopropylimidazole, N-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine, biotin, hydantoin and benzimidazole, imidazole being particularly preferred; amino acids such as in particular arginine, citrulline, histidine, ornithine, lysine, oligopeptides consisting of on average 2 to 3 amino acids having a high proportion (>50%, in particular >70%) of the cited amino acids, and salts thereof, preferably arginine and salts thereof and arginine-rich oligopeptides; diols such as for example 2-ethyl-1,3-hexanediol, 1,3-butanediol, 1,4-butanediol, 1,2-propanediol, 1,3-propanediol, neopentyl glycol and ethylene glycol, with 1,3-diols, in particular 2-ethyl-1,3-hexanediol and 1,3-butanediol, being particularly preferred.

With regard to more detailed information about such components to strengthen the permanent wave capacity, reference is made to the publications DE-OS 44 36 065 and EP-BI-363 057.

Compounds for strengthening the permanent wave capacity can be included in permanent wave lotions according to the invention in amounts from 0.5 to 5 wt. %, relative to the complete permanent wave lotion. Amounts from 1 to 4 wt. %, in the case of diols from 0.5 to 3 wt. %, have proved to be adequate, for which reason these amounts are particularly preferred.

Oxidizing agents such as sodium bromate, potassium bromate, hydrogen peroxide, and conventional stabilizers for stabilizing aqueous hydrogen peroxide preparations are a mandatory constituent of fixing agents according to the invention. Aqueous H₂O₂ preparations contain approximately 0.5 to 15 wt. %, generally approximately 0.5 to 3 wt. % of hydrogen peroxide in the ready-to-use state. The pH of such aqueous H₂O₂ preparations is preferably around 2 to 6, in particular 2 to 4, and is established by means of inorganic acids, preferably phosphoric acid. Bromate-based fixing agents typically contain bromates in concentrations of 1 to 10 wt. % with the pH of the solutions adjusted to 4 to 7. Fixing agents on an enzymatic basis (e.g., peroxidases) having little or no oxidizing agent, particularly hydrogen peroxide, are likewise suitable.

Permanent wave agents or fixing agents according to the invention are conventionally formulated as a single phase; this term includes systems having a continuous phase, such as pure oil-in-water or water-in-oil emulsions. It has been found that two-phase and multi-phase systems are also preferred according to the invention. These are systems in which at least two separate, continuous phases are present.

It is further advantageous for the oxidizing agent preparations comprising oxidation dyes, lightening agents and/or fixing agents of shape-changing agents to contain at least one stabilizer or complexing agent. Common preferred chelating agents include polycarboxylic acids, nitrogen-containing mono- or polycarboxylic acids, particularly ethylenediamine tetraacetic acid (EDTA), ethylenediamine disuccinic acid (EDDS) and nitrilotriacetic acid (NTA), geminal diphosphonic acids, particularly 1-hydroxyethane-1,1-diphosphonic acid (HEDP), aminophosphonic acids such as ethylenediamine tetra(methylene phosphonic acid) (EDTMP), diethylenetriamine penta(methylene phosphonic acid) (DTPMP), phosphonopolycarboxylic acids such as 2-phosphonobutane-1,2,4-tricarboxylic acid, and cyclodextrins, alkali stannates (sodium stannate), alkali pyrophosphates (tetrasodium pyrophosphate, disodium pyrophosphate), alkali phosphates (sodium phosphate), and phosphoric acid.

Agents according to the invention can contain further auxiliary substances and additives. The ready-to-use agents are preferably free-flowing preparations. These free-flowing preparations preferably also contain an emulsifier or surfactant as a surface-active substance, surface-active substances being referred to as surfactants or as emulsifiers depending on the area of application and being selected from anionic, cationic, amphoteric, zwitterionic and non-ionic surfactants and emulsifiers. These substances are described in detail below.

All anionic surface-active substances suitable for use on the human body are suitable as anionic surfactants in preparations according to the invention. These have a water-solubilizing anionic group such as a carboxylate, sulfate, sulfonate or phosphate group and a lipophilic, saturated or unsaturated alkyl group having approximately 8 to 30 C atoms and 0, 1, 2 or 3 double bonds. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are
linear and branched, saturated and unsaturated fatty acids (soaps),
optionally polyalkoxylated ether carboxylic acids of the formula RO(CH₂CH₂O)ₓCH₂COOH, in which R is a linear alkyl group and x=0 or a number from 1 to 16,
acyl sarcosides,
acyl taurides,
acyl isethionates,
sulfosuccinic acid mono- and dialkyl esters and sulfosuccinic acid monoalkyl polyoxyethyl esters and 1 to 6 oxyethyl groups,
linear alkane sulfonates,
linear α-olefin sulfonates,
sulfonates of unsaturated fatty acids,
α-sulfo fatty acid methyl esters of fatty acids,
alkyl sulfates and alkyl ether sulfates of the formula RO(CH₂CH₂O)ₓSO₃H, in which R is a preferably linear alkyl group and x=0 or a number from 1 to 12,
mixtures of surface-active hydroxyl sulfonates, sulfated hydroxyalkyl polyethylene and/or hydroxyalkylene propylene glycol ethers,
esters of tartaric acid and citric acid with alcohols which are addition products of around 2 to 15 molecules of ethylene oxide and/or propylene oxide with fatty alcohols,
alkyl and/or alkenyl ether phosphates of the formula

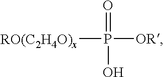

in which R preferably is an aliphatic, optionally unsaturated, hydrocarbon residue having 8 to 30 carbon atoms, R′ is hydrogen, a (CH₂CH₂O)_{y}R residue, and x and y are each independently a number from 1 to 10,
sulfated fatty acid alkylene glycol esters of the formula RC(O)O(alkO)ₙSO₃H, in which R is a linear alkyl residue, alk is CH₂CH₂, CHCH₃CH₂ and/or CH₂CHCH₃ and n is a number from 0.5 to 5,
monoglyceride sulfates and monoglyceride ether sulfates.

Preferred anionic surfactants are alkyl sulfates, alkyl ether sulfates, each having 10 to 18 C atoms in the alkyl group, and polyalkoxylated ether carboxylic acids having 10 to 18 C atoms in the alkyl group and up to 12 glycol ether groups in the molecule.

Surface-active compounds classified as zwitterionic surfactants are those bearing at least one quaternary ammonium group and at least one carboxylate, sulfonate or sulfate group in the molecule. Particularly suitable zwitterionic surfactants are betaines such as N-alkyl-N,N-dimethylammonium glycinates, for example, cocoalkyl dimethylammonium glycinate, N-acyl aminopropyl-N,N-dimethylammonium glycinates, for example, cocoacylaminopropyl dimethylammonium glycinate, and 2-alkyl-3-carboxylmethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

In a further, preferred embodiment the agent contains at least one amphoteric surfactant. Amphoteric surfactants are surface-active compounds which, in addition to a C₈-C₂₄ alkyl or acyl group, contain at least one free amino group and at least one —COOH or —SO₃H group in the molecule and are capable of forming internal salts. Examples of suitable amphoteric surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred amphoteric surfactants are marketed under the INCI name Disodium Cocoamphodipropionate with the trade names Miranol C2M SF conc. (Rhodia), Amphoterge K-2 (Lonza) and Monateric CEM-38 (Unichema) and under the name Disodium Cocoamphodiacetate with the trade names Dehyton (Cognis), Miranol C2M (Rhodia) and Ampholak XCO 30 (Akzo Nobel).

The addition in the agent brings about an outstanding finishing effect, in particular improved wet combability.

It has also proved advantageous for lightening agents according to the invention to contain additional, non-ionogenic interfacially-active substances in addition to the polyalkoxylated fatty substances described above. Such compounds include C₁₂-C₃₀ fatty acid mono- and diesters of addition products of 1 to 30 mol of ethylene oxide with glycerol; polyglycerol esters and alkoxylated polyglycerol esters, such as for example poly(3)glycerol diisostearate (commercial product: Lameform®TGI (Henkel)) and poly(2)glycerol polyhydroxystearate (commercial product: Dehymuls®PGPH (Henkel)); alkoxylated, preferably propoxylated and in particular ethoxylated, mono-, di- and triglycerides, such as glycerol monolaurate+20 EO (mol of ethylene oxide) and glycerol monostearate+20 EO; amine oxides; sorbitan fatty acid esters and addition products of ethylene oxide with sorbitan fatty acid esters such as polysorbates, sorbitan monolaurate and sorbitan monolaurate+20 EO; sugar fatty acids and addition products of ethylene oxide with sugar fatty acid esters; addition products of ethylene oxide with fatty acid alkanol amides and fatty amines; fatty acid-N-alkyl glucamides; alkyl phenols and alkyl phenol alkoxylates having 6 to 21, particularly 6 to 15 carbon atoms in the alkyl chain and 0 to 30 ethylene oxide and/or propylene oxide units, such as nonyl phenol+4 EO, nonyl phenol+9 EO, octyl phenol+3 EO and octyl phenol+8 EO as well as alkyl polyglycosides corresponding to the general formula RO—(Z)ₓ, in which R is alkyl, Z is sugar and x is the number of sugar units.

C₈ to C₂₂ alkyl mono- and oligoglycosides and ethoxylated analogs thereof are suitable as non-ionic surfactants. In particular, non-ethoxylated compounds have proved to be particularly suitable. Alkyl polyglycosides of the formula RO—(Z)ₓ, in which R consists substantially of C₈ and C₁₀ alkyl groups, C₁₂ and C₁₄ alkyl groups, C₈ to C₁₆ alkyl groups, C₁₂ to C₁₆ alkyl groups or C₁₆ to C₁₈ alkyl groups, are particularly preferred. Any mono- or oligosaccharides can be used as the sugar structural unit Z. Sugars having 5 or 6 carbon atoms and the corresponding oligosaccharides are typically used. Such sugars include glucose, fructose, galactose, arabinose, ribose, xylose, lyxose, allose, altrose, mannose, gulose, idose, talose and sucrose. Preferred sugar structural units are glucose, fructose, galactose, arabinose and sucrose; glucose is particularly preferred. The alkyl polyglycosides for use according to the invention contain on average 1.1 to 5 sugar units. Alkyl polyglycosides having x values from 1.1 to 2.0 are preferred. Alkyl glycosides in which x is 1.1 to 1.8 are most particularly preferred.

Anionic, additional non-ionic, zwitterionic and amphoteric surfactants are preferably used in a total amount from 0.1 to 45 wt. %, preferably 1 to 30 wt. % and most particularly preferably 1 to 20 wt. %, relative to the overall amount of the ready-to-use agent.

Likewise preferred are cationic surfactants of the quaternary ammonium compound, esterquat and amidoamine type. Preferred quaternary ammonium compounds include ammonium halides, particularly chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI names Quaternium-27 and Quaternium-83. The long alkyl chains of the aforementioned surfactants preferably have 10 to 18 carbon atoms. Other cationic surfactants which can be used are the quaternized protein hydrolysates.

Likewise advantageous according to the invention are quaternary ester compounds known as esterquats. Esterquats are known substances containing both at least one ester function and at least one quaternary ammonium group as a structural element. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines, such as N,N-bis-(2-palmitoyloxyethyl)dimethyl ammonium chloride. Such products are sold, for example, under the trademarks Stepantex, Dehyquart and Armocare.

Cationic surfactants can be present in agents according to the invention in amounts from 0.05 to 10 wt. %, relative to the complete agent. Amounts from 0.1 to 5 wt. % are particularly preferred.

It has proved advantageous if the agents contain at least one thickening agent. There are no restrictions in principle in regard to these thickening agents. Both organic and purely inorganic thickening agents can be used.

The thickening agent is preferably an anionic, synthetic polymer. Preferred anionic polymers are acrylic acid and/or methacrylic acid polymers or copolymers, preferably present in the agents in an amount from 0.1 to 10 wt. %, particularly preferably from 1 to 5 wt. %, relative in each case to the weight of the agent. The thickening agent is furthermore preferably a cationic synthetic polymer. Preferred cationic groups are quaternary ammonium groups. In particular, polymers in which the quaternary ammonium group is bonded via a C₁ to C₄ hydrocarbon group to a polymer main chain synthesized from acrylic acid, methacrylic acid or derivatives thereof have proved to be particularly suitable. Such polymers can also be used as copolymers with non-ionogenic monomer units, preferably acrylamide, methacrylamide, acrylic acid C₁-C₄ alkyl esters and methacrylic acid C₁-C₄ alkyl esters. Non-ionic, fully synthetic polymers, such as for example polyvinyl alcohol or polyvinyl pyrrolidinone, can likewise be used as thickening agents according to the invention. Naturally occurring, optionally modified thickening agents are also preferably used. These include guar gums, scleroglucan gums or xanthan gums, vegetable gums, such as gum arabic, ghatti gum, karaya gum, tragacanth gum, carrageen gum, agar-agar, carob seed meal, pectins, alginates, starch, starch fractions and derivatives such as amylose, amylopectin and dextrins, cellulose derivatives such as methylcellulose, carboxyalkyl celluloses and hydroxyalkyl celluloses. Layered silicates (polymeric, crystalline sodium disilicates) have proved to be particularly suitable within the meaning of the present invention as inorganic thickening agents. Clays, in particular, magnesium aluminum silicates such as bentonite, particularly smectites, such as montmorillonite or hectorite, which can optionally also be suitably modified, and synthetic layered silicates are preferred in particular.

In a preferred embodiment the agents additionally contain at least one further active ingredient which improves the caring properties of the agents in a synergistic manner. This active ingredient is preferably chosen from amino acids, oligopeptides and protein hydrolysates; silicone derivatives; polyphenols and (pseudo)ceramides.

In a preferred embodiment the agent additionally contains at least one further active ingredient chosen from amino acids, oligopeptides and protein hydrolysates. Within the meaning of the invention, amino acids and oligomers and polymers thereof coupled by peptic bonds, in particular oligopeptides and protein hydrolysates, are to be understood as the active ingredient. An amino acid within the meaning of the invention is classified as an organic compound bearing in its structure at least one protonizable amino group and at least one carboxylic acid or a sulfonic acid group. Preferred amino acids are aminocarboxylic acids, particularly α-aminocarboxylic acids and ω-aminocarboxylic acids, α-aminocarboxylic acids being preferred. The active ingredient consisting of at least one amino acid, an oligopeptide or a protein hydrolysate is included in the agents in amounts of preferably 0.01 to 10 wt. %, particularly 0.05 to 5 wt. %, relative to the overall weight of the application mixture.

Preferred silicone derivatives are functionalized and non-functionalized, optionally branched polyalkyl siloxanes, polyaryl siloxanes, polyalkylaryl siloxanes, such as dimethicones, amodimethicones, cyclomethicones and dimethicone copolyols. Preferred agents can contain silicone derivatives in amounts of 0.01 to 5 wt. %, preferably 0.05 to 2 wt. %, relative in each case to the ready-to-use agent.

Polyphenols are generally compounds containing more than two phenol (polyol) or phenol ether groups belonging to different classes of substances. Polyphenols are preferably chosen from at least one representative of the group hydroxycinnamic acids, 6,7-dihydroxycoumarines, hydroxybenzoic acids, catechins, leucoanthocyanidins, anthocyanidins, flavanones, flavones and flavonols.

Sphingolipids are preferably used as ceramides. Preferred compounds used as the active ingredient are compounds known under the INCI names Ceramide 1, Ceramide II, Ceramide 1, Ceramide 2, Ceramide 3, Ceramide 5 and Ceramide 6. Mixtures of such compounds which are available for example from Degussa Care Specialties under the trade names SK-Influx and Ceramide III and the commercial product Ceramide TIC-001 marketed by Takasago International Corporation are particularly preferably used. These compounds are preferably used in an amount from 0.01 to 1.0 wt. %, relative to the weight of the ready-to-use cosmetic agent.

Agents according to the invention can furthermore contain additional active ingredients, auxiliary substances and additives, such as non-ionic polymers, cationic polymers, zwitterionic and amphoteric polymers, anionic polymers, texturizing agents such as glucose, maleic acid and lactic acid, hair-conditioning compounds such as phospholipids, active ingredients to improve the fiber structure, particularly mono-, di- and oligosaccharides such as glucose, galactose, fructose, fruit sugar and lactose, defoaming agents such as silicones, preferably dimethicone, dyes to color the agent, anti-dandruff active ingredients, light stabilizers, active ingredients such as panthenol, pantothenic acid, pantolactone, allantoin, pyrrolidinone carboxylic acids and salts thereof as well as bisabolol, vitamins, provitamins and vitamin precursors, in particular those of groups A, B₃, B₅, B₆, C, E, F and H, plant extracts, fats and waxes such as spermaceti wax, beeswax, montan wax and paraffins, swelling and penetrating substances such as glycerol, propylene glycol monoethyl ether, carbonates, hydrogen carbonates, guanidines, ureas as well as primary, secondary and tertiary phosphates, opacifiers such as latex, styrene/PVP and styrene/acrylamide copolymers, pearlescent agents such as ethylene glycol mono- and distearate and PEG-3 distearate, pigments, blowing agents such as propane-butane mixtures, N₂O, dimethyl ether, CO₂ and air and antioxidants.

The person skilled in the art selects these additional substances according to the desired properties of the agents. Regarding further optional components and the amounts used, reference is expressly made to the relevant manuals known to the person skilled in the art, for example, Kh. Schrader, Grundlagen and Rezepturen der Kosmetika, 2^{nd} Edition, Hüthig Buch Verlag, Heidelberg, 1989.

Preferred agents for changing the color and shape of the hair have the characteristic feature of having as neutral a pH as possible. A further preferred embodiment consists in that the ready-to-use agent has a pH of from 4.0 to 12.0, preferably from 5.0 to 11.5, more preferably from 6.0 to 11.0. The pH values according to the present invention are pH values measured at a temperature of 22° C. The pH is conventionally adjusted with pH adjusters. Acidifying and alkalizing agents commonly used in cosmetics for adjusting the pH are familiar to one skilled in the art. Alkalizing agents which can be used for adjusting the pH are typically chosen from inorganic salts, particularly alkali and alkaline-earth metals, organic alkalizing agents, in particular amines, basic amino acids and alkanol amines, and ammonia. Preferred acidifying agents according to the invention are food acids such as citric acid, acetic acid, malic acid or tartaric acid, as well as dilute mineral acids.

Preferred alkalizing agents are chosen from sodium hydroxide, potassium hydroxide, calcium hydroxide, barium hydroxide, sodium phosphate, potassium phosphate, sodium silicate, potassium silicate, sodium carbonate, potassium carbonate, 2-aminoethan-1-ol (monoethanolamine), triethanolamine, ammonia, 1-aminopropan-2-ol and 2-amino-2-methyl propan-1-ol.

The packaging of the color- and shape-changing agents according to the invention is not subject in principle to any restrictions. Agents according to the invention are typically packaged as one-component agents which, for example, are optionally mixed immediately before application with a second preparation containing an oxidizing agent. In some cases, however, it is preferable for the product to be packaged as a two-component agent. In a preferred embodiment the agents are therefore packaged in such a way that one of the components substantial to the invention is packaged separately. According to the invention it is initially irrelevant which of the components according to the invention is packaged separately. It can however be preferable to package the preparation containing the animal-based care component separately until the time of its application.

Preferred agents according to the invention are prepared directly before application by mixing at least two preparations. These at least two preparations can be provided in at least two separately packaged containers, with one container having a coloring agent containing in a cosmetic carrier at least one oxidation dye precursor and at least one polyalkoxylated fatty substance as well as at least one animal-based care component, preferably royal jelly, and which has a pH of from 5.0 to 12.0, preferably from 6.0 to 11.0, and a further container having an oxidizing agent preparation containing at least one oxidizing agent.

The present invention also provides a method for changing the color and/or permanently changing the shape of keratinic fibers, wherein an agent according to the invention is applied to the hair in accordance with the above instructions, left on the hair for a contact period of 2 to 45 minutes, preferably 15 to 30 minutes, and is then rinsed out of the hair.

In a preferred embodiment of the method for changing the color of keratinic fibers, a composition in a cosmetic carrier containing at least one oxidation dye precursor and at least one polyalkoxylated fatty substance as well as at least one animal-based care component, preferably royal jelly, is mixed with an oxidizing agent preparation containing hydrogen peroxide to form a homogeneous composition, and this is applied to the hair, left on the hair for a contact period of 2 to 45 minutes, preferably 15 to 30 minutes, and then rinsed out of the hair. The application temperatures for the color changing according to the invention of keratinic fibers can be in a range from 15 to 45° C. After a contact period the hair coloring agent is removed from the hair to be colored by rinsing. There is no need to wash with a shampoo afterwards if a carrier with high surfactant content (e.g., a coloring shampoo) was used.

In a further preferred embodiment of the method for the permanent shape changing of keratinic fibers, a composition in a cosmetic carrier containing at least one keratin-reducing substance and at least one polyalkoxylated fatty substance as well as at least one animal-based care component, preferably royal jelly, is applied to the hair, left on the hair for a contact period of 2 to 45 minutes, preferably 15 to 30 minutes, and then optionally rinsed out of the hair. A fixing agent containing at least one oxidizing agent, preferably hydrogen peroxide, and preferably additionally at least one stabilizer or complexing agent is then applied to the hair, left on the hair for a contact period of 2 to 45 minutes, preferably 15 to 30 minutes, and then rinsed out of the hair. The application temperatures for the permanent shape changing according to the invention of keratinic fibers can be in a range between 15 and 45° C. After a contact period the fixing agent is removed from the hair by rinsing. There is no need to wash with a shampoo afterwards if a carrier with high surfactant content was used.

All that has been stated regarding the agents according to the invention applies with necessary alterations to further preferred embodiments of the method according to the invention.

As has already been mentioned, the agents can also be prepared directly before application from two or more separately packaged preparations. In particular, this allows the separation of incompatible constituents thereby preventing a premature reaction. In such systems the ready-to-use agent is prepared by the consumer by mixing the components directly before application. A coloring and/or lightening agent in which the oxidation dye precursors are initially separate from the oxidizing agent preparation preferably containing hydrogen peroxide is preferred here.

The present invention therefore also provides a kit of parts containing at least two separately packaged containers, one container containing at least one color- and/or shape-changing compound and at least one polyalkoxylated fatty substance as well as at least one animal-based care component, preferably royal jelly, and one container containing an oxidizing agent composition containing at least one chemical oxidizing agent, in particular hydrogen peroxide.

A preferred embodiment is therefore a kit of parts containing at least two separately packaged containers, one container containing a coloring mixture in a cosmetic carrier containing at least one coloring component, particularly at least one oxidation dye precursor, at least one polyalkoxylated fatty substance as well as at least one animal-based care component, preferably royal jelly, and one container containing an oxidizing agent composition containing at least one chemical oxidizing agent, particularly hydrogen peroxide.

If a particularly intense lightening effect is desired through the use of persulfate salts or peroxodisulfate salts, it is preferable for these to be included with the kit of parts according to the invention as a separately packaged, additional component in the form of an optionally dedusted powder or a pressed molding.

A further preferred embodiment is a kit of parts containing at least two separately packaged containers, one container containing a shape-changing mixture in a cosmetic carrier containing at least one shape-changing component and at least one polyalkoxylated fatty substance as well as at least one animal-based care component, preferably royal jelly, and one container containing an oxidizing agent composition containing at least one chemical oxidizing agent, particularly hydrogen peroxide.

It has furthermore likewise been found that the damage to hair by color- and/or shape-changing agents can be reduced and its condition improved if, subsequent to a color and/or shape change, the hair is treated with a post-treatment agent containing at least one polyalkoxylated fatty substance and at least one animal-based care component, preferably royal jelly. The term “subsequent to” refers to the period of up to two hours, preferably up to one hour immediately after completion of the color- and/or shape-changing treatment. It has been found that if the post-treatment preparation is used at a later time (e.g., more than two hours after changing the color and/or shape of the hair with this post-treatment agent), the reduction of damage to the hair and care performance by the post-treatment agent is greatly reduced or no longer perceptible at all.

The present invention therefore also provides a kit of parts containing at least two separately packaged containers, one container having at least one color- and/or shape-changing preparation and one container having at least one post-treatment agent containing at least one polyalkoxylated fatty substance as well as at least one animal-based care component, preferably royal jelly.

All that has been stated in respect of the agents according to the first subject-matter of the invention applies with necessary alterations to particular embodiments of the color- and/or shape-changing component, the polyalkoxylated fatty substances and the animal-based care component and of further additives in color- and/or shape-changing preparations and in the post-treatment agent.

In a particular embodiment with regard to shape-changing agents or oxidative color-changing agents, preferably the kit of parts additionally contain, in addition to a container having at least one color- and/or shape-changing preparation and a container having at least one post-treatment agent containing at least one polyalkoxylated fatty substance and at least one animal-based care component, preferably royal jelly, a container having an oxidizing agent composition containing at least one chemical oxidizing agent, particularly hydrogen peroxide. The preferred embodiments described above apply with necessary alterations to the oxidizing agent preparation.

The kit of parts preferably includes instructions for use which explain the sequence of use and optionally the sequence of mixing the individual preparations. It can moreover be preferable if an application aid such as a comb or a brush, and/or personal protection equipment such as disposable gloves are also included with the kit. All that has been stated in respect of the agents according to the invention applies with necessary alterations to further preferred embodiments of the kit of parts.

The invention also provides for use of the agents of the first subject-matter of the invention to reduce damage to the hair when changing the color and/or permanently changing the shape of human hair. Finally the invention also provides the use of the agents of the first subject-matter of the invention to change the color and/or permanently change the shape of human hair with reduced damage to the hair.

The invention also provides for use of an agent prepared by mixing the components of a kit of parts to reduce damage to the hair when changing the color and/or permanently changing the shape of human hair.

Finally the invention also provides for use of the components of a kit of parts in the aforementioned time sequence to reduce damage to the hair when changing the color and/or permanently changing the shape of human hair.

All that has been stated in respect of the agents according to the invention applies with necessary alterations to further preferred embodiments of the uses according to the invention.

The examples below are intended to clarify the present invention without restricting its scope.

### EXAMPLES

### I.

### 1) Coloring Creams

| Raw material | E1 | E2 | E3 | E4 | E5 | E6 |
|---|---|---|---|---|---|---|
| Lanette D | 6.60 | 6.60 | 6.60 | 6.60 | 6.60 | 5.50 |
| Lorol techn. | 2.40 | 2.40 | 2.40 | 2.40 | 2.43 | 2.00 |
| Eumulgin B2 | 0.60 | 0.60 | 0.60 | 0.60 | 0.60 | 0.50 |
| Eumulgin B1 | 0.60 | 0.60 | 0.60 | 0.60 | 0.60 | 0.50 |
| Lamesoft PO 65 | 2.00 | 2.00 | 2.00 | 2.00 | 2.00 | 2.00 |
| Akypo Soft 45HP | 10.00 | 10.00 | 10.00 | 10.00 | 10.00 | 10.00 |
| Texapon K 14 S Special, 70% | 2.80 | 2.80 | 2.80 | 2.80 | 2.80 | 2.80 |
| Product W 37 194 | 3.75 | 3.75 | 3.75 | 3.75 | 3.75 | 3.75 |
| p-Toluylene diamine sulfate | 0.300 | 0.822 | 0.029 | — | 1.533 | 1.161 |
| Resorcinol | 0.350 | 0.416 | 0.011 | — | 0.410 | 0.298 |
| 2-Methylresorcinol | 0.246 | — | — | 0.004 | 0.343 | 0.128 |
| 5-Amino-2-methylphenol | — | 0.023 | 0.001 | — | — | — |
| 4-Amino-3-methylphenol | 0.460 | 0.019 | — | — | — | — |
| 2-Chloro-6-methyl-3-aminophenol | — | 0.047 | — | — | — | — |
| Bis-(5-amino-2-hydroxyphenyl)methane | — | 0.539 | — | 0.010 | — | — |
| 2HCI | | | | | | |
| 2,4-Diaminophenoxyethanol 2HCI | — | — | 0.002 | — | — | — |
| 3-Aminophenol | — | — | 0.002 | — | — | 0.058 |
| 2,7-Dihydroxynaphthalene | — | — | — | 0.104 | — | — |
| 2,4,5,6-Tetraaminopyrimidine sulfate | — | — | — | 0.146 | — | — |
| 2-Amino-3-hydroxypyridine | — | — | — | — | 0.046 | 0.078 |
| Ammonium sulfate, techn. pure | 0.82 | 0.51 | 1.00 | 0.92 | — | 0.50 |
| Sodium sulfite, anhydrous, 96% | 0.40 | 0.40 | 0.40 | 0.40 | 0.40 | 0.40 |
| HEDP, 60% | 0.20 | 0.20 | 0.20 | 0.20 | — | 0.20 |
| Sodium hydroxide, 45% | 1.00 | 1.12 | 0.10 | 0.30 | 1.70 | — |
| Potassium hydroxide, 50% | — | — | — | — | — | 0.90 |
| Ascorbic acid | — | — | — | — | — | 0.05 |
| Sodium silicate 40/42 | 0.50 | 0.50 | 0.50 | 0.50 | 0.50 | 0.50 |
| L-Serine | 1.00 | 1.00 | 1.00 | 1.00 | 1.00 | — |
| Mellidyn LS 9657 | 1.00 | 1.00 | 1.00 | 1.00 | 1.00 | 1.00 |
| Ammonia, 25% | 7.00 | 6.50 | 7.00 | 7.00 | 6.50 | 7.50 |
| Perfume | qs | qs | qs | qs | qs | qs |

| | |
|---|---|
| Water, demineralized | to 100 |

| | |
|---|---|
| Raw materials used: | |
| Lanette D | C₁₆-C₁₈ fatty alcohol (INCI name: Cetearyl alcohol) (Cognis) |
| Lorol tech. | C₁₂-C₁₈ fatty alcohol (INCI name: Coconut alcohol) (Cognis) |
| Eumulgin B2 | C₁₆-C₁₈ fatty alcohol, ethoxylated (20 EO) (INCI name: Ceteareth- |
| | 20) (Cognis) |
| Eumulgin B1 | C₁₆-C₁₈ fatty alcohol, ethoxylated (12 EO) (INCI name: Ceteareth- |
| | 12) (Cognis) |
| Lamesoft PO 65 | C₁₂-C₁₈ alkylpolyglucoside, glyceryl monooleate (approx. 66%, |
| | INCI name: Coco-Glucoside, Glyceryl Oleate, Aqua) (Cognis) |
| Akypo Soft 45HP | C₁₂-C₁₄ alkyl ether, ethoxylated (6 EO) carboxylic acid, sodium |
| | salt (approx. 21%, INCI name: Sodium Laureth-6 Carboxylate, |
| | Aqua) (KAO) |
| Texapon K14 S Special | C₁₂-C₁₄ alkyl ether sulfate, ethoxylated (3 EO), sodium salt |
| | (approx. 70%, INCI name: Sodium Myreth Sulfate, Aqua) (Cognis) |
| Product W 37194 | Copolymer consisting of acrylic acid, sodium salt and trimethyl |
| | ammoniopropyl acrylamide chloride (approx. 20%, INCI name: |
| | Acrylamidopropyltrimonium Chloride/Acrylates Copolymer, Aqua) |
| | (Stockhausen) |
| Sodium silicate 40/42 | Sodium silicate |
| Mellidyn LS 9657 | Preparation of monosaccharides from honey with propolis extract, |
| | honey and royal jelly (INCI name: Aqua, Propylene Glycol, Mel |
| | Extract, Glycerin, PEG-40 Hydrogenated Castor Oil, Royal Jelly, |
| | Mel, Propolis) (Laboratoires Serobiologiques) |

Lanette D, Lorol, Eumulgin B2 and Eumulgin B1 and Lamesoft PO 65 were melted together at 80° C., dispersed with part of the water, the remaining constituents were incorporated, the mixture topped up with water and the formulation stirred until cold.

The ready-to-use coloring agents are prepared by mixing equal percentages by weight of the individual cream (E1 to E5) with the following developer dispersion:

| Raw material | Weight [wt. %] |
|---|---|
| Dipicolinic acid | 0.10 |
| Disodium pyrophosphate | 0.03 |
| HEDP, 60% | 1.50 |
| Texapon NSO | 2.00 |
| Dow Corning DB 110 A | 0.07 |
| Aculyn 33A | 15.00 |
| Ammonia, 25% | 0.65 |
| Water, demineralized | to 100 |

Texapon NSO UP Lauryl alcohol diglycol ether sulfate, Na salt (approx. 28%, INCI name: Sodium Laureth Sulfate) (Cognis)
Aculyn 33 Acrylic polymer (approx. 28% in water; INCI name: Acrylates Copolymer) (Rohm & Haas)
Dow Corning DB 110 Non-ionic silicone emulsion (INCI name: Dimethicone) (Dow Corning)

### 2) Pigment Removal:

4 g of the freshly prepared, ready-to-use coloring agent were applied per gram of hair (European human hair, Alkinco 6634, #10/2003, A9). The contact period for the coloring agents was 30 minutes at 35° C. The strands were then rinsed with warm water for 30 seconds and then air dried. The colored strands had glossy colors and a pleasant texture.

II.

### 1) Coloring Cream

| Raw materials | E1 |
|---|---|
| powdered dye blend* | 2.33 |
| Ammonium carbomer, 1% | 12.00 |
| Lanette E, powder | 0.56 |
| Texapon NSF, 27% | 3.52 |
| Potassium oleate, 12.5% | 2.40 |
| Cutina GMS SE | 1.60 |
| Cutina AGS | 1.60 |
| Eutanol G | 1.60 |
| Hydrenol D | 9.60 |
| Eumulgin B2 | 2.40 |
| L-Serine | 0.50 |
| Phospholipid EFA | 0.10 |
| Na₄-EDTA, powder, 87% | 0.20 |
| Hydrovance | 2.00 |
| Herbasol extract aloe, decolorized | 1.00 |
| Mellidyn LS 9657 | 1.00 |
| Ascorbic acid | 0.05 |
| Sodium sulfite, anhydrous, 96% | 0.20 |
| Ammonia, 25% | 6.00 |
| Potassium hydroxide, 50% | 0.85 |
| Perfume | qs |
| Water | to 100 |

*Blend of dyes containing 59.1 wt. % p-toluylene diamine sulfate, 6.6 wt. % 3-aminophenol, 22.7 wt. % resorcinol, 0.8 wt. % 2-amino-4-hydroxyethyl aminoanisol sulfate and 10.8 wt. % silicon dioxide, pyrogenic, relative in each case to the overall weight of the dye blend.
Raw materials used:
Lanette E, powder C₁₆-C₁₈ fatty alcohol sulfate, sodium salt (INCI name: Sodium Cetearyl Sulfate) (Cognis)
Texapon NSF, 27% C₁₂ fatty alcohol sulfate, ethoxylated (2 EO) sodium salt (INCI name: Sodium Laureth Sulfate) (Cognis)
Cutina GMS SE Stearic acid glyceryl ester (INCI name: Glyceryl Stearate) (Cognis)
Cutina AGS Ethylene glycol distearyl ester (INCI name: Glycol Distearate) (Cognis)
Eutanol G 2-Octyldodecanol (INCI name: Octyldodecanol) (Cognis)
Hydrenol D C₁₆-C₁₈ fatty alcohol (INCI name: Cetearyl alcohol) (Cognis)
Eumulgin B2 C₁₆-C₁₈ fatty alcohol, ethoxylated (20 EO) (INCI name: Ceteareth-20) (Cognis)
Phospholipid EFA Zwitterionic phospholipid (INCI name: Linoleamidopropyl PG-dimonium chloride phosphate) (Uniqema)
Hydrovance Hydroxyethyl urea (approx. 50%; INCI name: Hydroxyethyl urea, Aqua) (National Starch)
Herbasol extract aloe Water/propylene glycol extract of Aloe Vera (2-4% active substance; INCI name: Aqua, Propylene Glycol, Aloe Barbadensis)
(Cosmetochem)

The fat base was melted together at 80° C. and dispersed with part of the water. The remaining formulation ingredients were then incorporated in sequence while stirring. The mixture was then made up with water to 100 wt. % and the formulation stirred until cold.

The ready-to-use coloring cream was mixed in a ratio of 1:1 with a developer dispersion having a composition as follows.

| Raw material | wt. % |
|---|---|
| Na benzoate | 0.04 |
| Dipicolinic acid | 0.10 |
| Disodium pyrophosphate | 0.19 |
| 1,2-Propanediol | 0.50 |
| HEDP, 60% | 0.25 |
| Paraffinum Liquidum | 0.30 |
| Genamin STAC | 0.20 |
| Cetearyl alcohol | 3.00 |
| Eumulgin B 2 | 0.70 |
| Hydrogen peroxide 50% | 12.2 |
| Potassium hydroxide, 50% | 0.19 |
| Water | to 100 |

Genamin STAC Trimethyl stearyl ammonium chloride (approx. 80% active substance; INCI name: Steartrimonium Chloride) (Clariant)

### 2) Pigment Removal:

For the coloring process 4 times the amount of finished application mixtures was applied to strands of bleached buffalo belly hair weighing approx. 0.7 g. After the strands had been colored for 30 minutes at 32° C., they were washed with a commercial shampoo and dried with a hairdryer. The colored strands had glossy colors and a pleasant texture.

We claim:

1. Agent for changing the color and/or shape of keratinic fibers comprising in a cosmetic carrier:
at least one color- and/or shape-changing component,
at least one polyalkoxylated fatty substance, and
at least one animal-based care component.

2. Agent according to claim 1, wherein the animal-based care component is royal jelly.

3. Agent according to claim 2, wherein the royal jelly is present in an amount of from 0.00001 to 5%, based on total weight of the ready-to-use agent.

4. Agent according to claim 1, wherein the polyalkoxylated fatty substance is chosen from addition products of polyethylene oxide with fatty alcohols and/or hydroxyl group-containing fatty acid triglycerides and from mono- or polyglycerylated fatty alcohols.

5. Agent according to claim 1, wherein the polyalkoxylated fatty substance is a polyalkoxylated, hydroxyl group-containing fatty acid triglyceride.

6. Agent according to claim 5, wherein the polyalkoxylated, hydroxyl group-containing fatty acid triglyceride is an addition product of polyethylene oxide with hydrogenated and/or unhydrogenated castor oil.

7. Agent according to claim 1, wherein the polyalkoxylated fatty substance is an addition product of polyethylene oxide with hydrogenated castor oil having an average degree of ethoxylation of from 1 to 100.

8. Agent according to claim 1, wherein the polyalkoxylated fatty substance is present in an amount of from 0.001 to 20 wt. %, based on total weight of the ready-to-use agent.

9. Agent according to claim 1 further comprising a water-soluble, organic solvent having at least two hydroxyl groups.

10. Agent according to claim 1, wherein the water-soluble, organic solvent having at least two hydroxyl groups is chosen from 1,2-ethanediol, 1,2-propanediol, 1,3-propanediol, 1,2-butanediol, 1,3-butanediol, 1,4-butanediol, 2,3-butanediol, 1,6-hexanediol, 2-(2-hydroxyethoxy)ethanol and glycerol.

11. Agent according to claim 1, wherein the color-changing component the agent further comprises at least one oxidation dye precursor and/or at least one substantive dye and/or at least one precursor of nature-analogous dyes and/or at least one lightening agent.

12. Agent according to claim 1, wherein the shape-changing component the agent further comprises at least one keratin-reducing substance.

13. Agent according to claim 1 further comprising at least one surface-active compound chosen from anionic, zwitterionic, amphoteric and non-ionic surfactants.

14. Agent according to claim 1 further comprising an oxidizing agent chosen from hydrogen peroxide and its solid addition products with organic and inorganic compounds.

15. Method of reducing oxidative damage of human hair comprising applying an agent according to claim 1 to the hair when changing the color and/or shape of the hair.

16. Kit of parts for changing the color and/or shape of keratinic fibers comprising:
a container comprising at least one color- and/or shape-changing compound, at least one polyalkoxylated fatty substance, and at least one animal-based care component, and
a container comprising an oxidizing agent composition containing at least one chemical oxidizing agent,
wherein the containers are separately packaged.

17. Kit of parts comprising at least two separately packaged containers, wherein one container comprises at least one color- and/or shape-changing preparation, and one container comprises at least one post-treatment agent having at least one polyalkoxylated fatty substance and at least one animal-based care component.
